(19) United States
(12) Patent Application Publication
(10) Pub. No.: US 2023/0128083 A1
(43) Pub. Date: Apr. 27, 2023

(54) ENDOSCOPIC ARTICULATION DEVICE

(71) Applicant: GYRUS ACMI, INC. D/B/A OLYMPUS SURGICAL TECHNOLOGIES AMERICA, Westborough, MA (US)

(72) Inventors: Spence Edward Cornia, Sandy, UT (US); Dustin Edward Gorringe, Riverton, UT (US); Justin Garth Tidwell, North Salt Lake, UT (US); Derek Alder Hutchison, Vineyard, UT (US); Blake Salgat, Park City, UT (US); Kelly David Christensen, Centerville, UT (US)

(21) Appl. No.: 18/046,999

(22) Filed: Oct. 17, 2022

Related U.S. Application Data
(60) Provisional application No. 63/262,890, filed on Oct. 22, 2021.
(60) Provisional application No. 63/268,668, filed on Feb. 28, 2022.

(51) Int. Cl. A61B 1/005; A61B 1/008

(52) U.S. Cl. CPC A61B 1/0052; A61B 1/0057; A61B 1/008; A61M 25/0136

(57) ABSTRACT

An articulating medical device with a handle and an articulating elongate member extending from the handle. The handle has a cavity with at least one first rotatable member. The first rotatable member has a first lever arm and a first termination coupling. Coupled to the termination coupling is at least one first articulation cable. The first lever arm is arranged to limit displacement of slack in the at least one first articulation cable when the at least one first articulation cable is not under tension.

### PRIORITY CLAIM

This application claims the benefit of priority to U.S. Provisional Patent Application No. 63/262,890, filed Oct. 22, 2021, and U.S. Provisional Patent Application No. 63/268,668, filed Feb. 28, 2022, the contents of which are incorporated by reference in their entireties.

### BACKGROUND

Endoscopic devices have an articulation component which allows the user to view, modify, repair, or otherwise interact with cavities and lumens within a human body. In some examples, endoscopes use articulation wires or cables to manipulate or control movement of an articulating insertion portion of an endoscope. For example, a pulley system having rotatable portions inside a handle or other control portion is used to guide, move or control the articulation wires or cables. For example, for a four-direction control there are two pulleys or rotatable portions, and respective components such as knobs as user-manipulated control mechanisms on the handle and wires within the handle. In such an example, a first cable articulates the insertion portion in a vertical direction and the second cable articulates the insertion portion in a horizontal direction. In other examples, the insertion portion is moved in one direction and the associated rotatable components only control one direction of movement, either horizontal or vertical.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of an exploded view of an articulating endoscopic device.

FIG. 2 illustrates an example of a rotatable member.

FIG. 3 illustrates an example of a movable member and rotatable member.

FIG. 4 illustrates an example of a movable member and rotatable member.

FIGS. 5A and 5B illustrates an example of two associated rotatable members.

FIGS. 6A-6F illustrate an example of the movement of rotatable member and the articulating cables.

### SUMMARY

In an example, endoscopic devices have articulation components which use wires or cables to control articulation of portions of the endoscopic device inserted into a lumen or cavity in a body. Such wires or cables can be coupled to rotatable members that transfer movement to the articulation portions. When the wires or cables are coupled to a rotatable member, insertion portions, can become disengaged with its associated pulley system and bow outward, or away, from the pulley system. When the wires or cables bow outward from the pulley system the wires or cables can become entangled with other wires or cables or other features within the endoscopic device. Therefore, a need has been identified to have a mechanism where the wires or cables of such an articulation component remain substantially inside or proximate to a track, path, guide or channel when the wire or cable is in use. For example, the wire or cable remain within a track, path, guide or channel when the wire or cable is under tension or slack. Such wires or cables undergo tension or slack when the associated mechanisms for movable the wire or cable are, for example, rotated causing the insertion portions to articulate or move in a desired direction.

In an example endoscopic device that uses wires or cables to control articulation of insertion portions, these articulation wires or cable (hereinafter “cable”) can become disengaged with an associated rotatable member, pulley system, articulation system or similar mechanism to move the cables. Therefore, a need has been identified to have a mechanism where the cables of an articulation component are coupled to a rotatable member or pulley system that provides some managed freedom, or slack, of the cables.

Cables in articulation devices, undergo tension or slack when the associated rotatable mechanisms move the cable. Rotation of rotatable members transfers movement to the insertion articulation portions of the endoscopic device. In an example, slack or tension in the cables translates a corresponding horizontal or vertical movement to the articulating member. The cables are coupled to the rotatable member, for example by threading, tying, heat staking, welding. In an example, the cables extend from the coupling portion, through tracks, paths, channels or the like and to the articulating member.

### DETAILED DESCRIPTION

This document describes, among other things, a device or mechanism to move an articulating feature of a medical device such as an endoscope or other medical device with an articulating component. The medical device associated with articulating features, in an example, has an articulating elongate component, associated control features and a handle. The handle of such a medical device is a hollow component, for example the handle has a cavity that contains mechanical and electrical components. Some of these mechanical or electrical components within a cavity of the handle (e.g. a housing) are mechanisms and components that transfer movement to an articulating elongate member at a distal end of the handle. An example component to transfer movement includes a rotatable member having lever arm (movable member) coupled to a surface of the rotatable member. Articulating cables can be coupled at a proximal end with the rotatable member, extend through the cavity of the handle and into the articulating elongate component. As the articulating cable extend from the rotatable member, the articulating cable passes through a path, guide, channel or other feature to limit displacement of the articulating cable. The movable member or lever arm is, for example, a component that limits displacement of slack in the articulating cable.

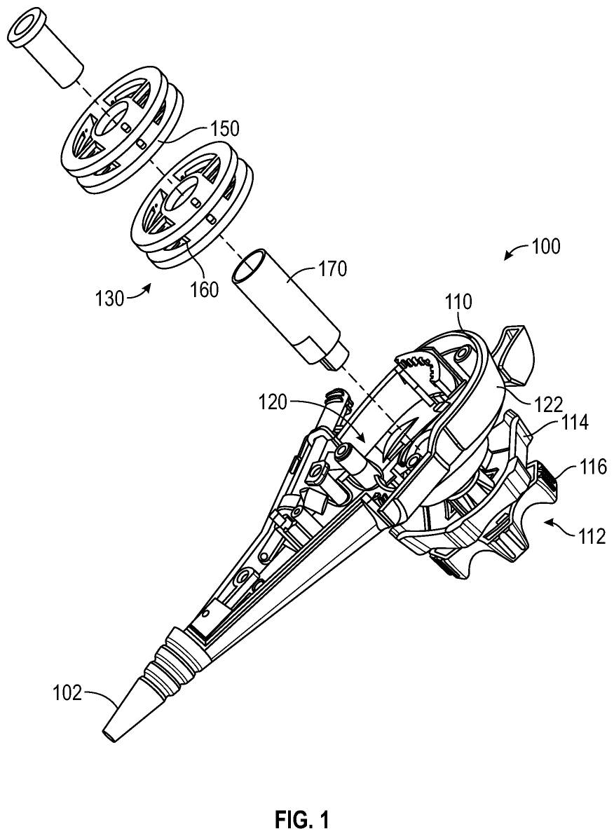

FIG. 1 illustrates an articulating medical device 100. The articulating medical device 100 is, for example, an endoscope with, for example, a handle 110 and an endoscopic component (articulating elongate member) 102 as an insertion portion. The components of the articulating medical device 100 control the movement of, for example, the articulating elongate member 102. The movement of the endoscopic component 102 can be controlled by an external rotatable control mechanism 112.

The rotatable control mechanism 112 is a user operated component to manipulate a rotatable member 130. The rotatable control mechanism 112 is external or on an exterior surface of the handle 110. The rotatable control mechanism 112, in an example, has a first rotatable control mechanism 114 (user manipulable control knob). The rotatable control mechanism 112 in another example has a second rotatable control mechanism 116 (user manipulable control knob). It is also contemplated to have more rotatable control mechanisms as dictated by the design.

As illustrated in FIG. 1, the handle 110 of the articulating medical device 100 includes a cavity 120 housing or containing at least electronic components and associated wiring, at least one articulating cable (hereinafter “cable”), and a rotatable member 130. The rotatable member 130 is, for example, a disc, reel, spool, pulley, cam-like feature or any similar device. The shape of the rotatable member 130 is one which allows efficient rotation such as circular, elliptical or otherwise eccentric.

The rotatable control mechanism 112 is coupled to the rotatable member 130. The rotatable member 130 has a shaft 170 extending through an outer wall 122 of the handle 110. The shaft 170 extends to the rotatable control mechanism 112 so the rotatable member 130 is coupled with the rotatable control mechanism 112. As a user operates the rotatable control mechanism 112, the rotatable member 130 moves in a reciprocal rotation. The rotatable control mechanism 112, in an example, rotates the rotatable member 130 bidirectionally about the center axis of rotation of the rotatable member 130. In an example where the rotatable member 130 controls four directional movement of the articulating elongate member 102, the first rotatable member 150 is coupled with the associated first rotatable control mechanism 114 or second rotatable control mechanism 116. The first rotatable control mechanism 114 transfers motion or rotation, either horizontal motion or vertical motion from rotation of the first rotatable control member 150 to the first rotatable member 150. The first rotatable control mechanism 114 or second rotatable control mechanism 116 then transfers a different motion or rotation, either horizontal motion or vertical motion, than the first rotatable control mechanism 114, to the second rotatable member 160. In an example, the rotatable member 130 with a first rotatable member 150 and a second rotatable member 160, the articulating elongate member 102 can be guided in horizontal and vertical directions.

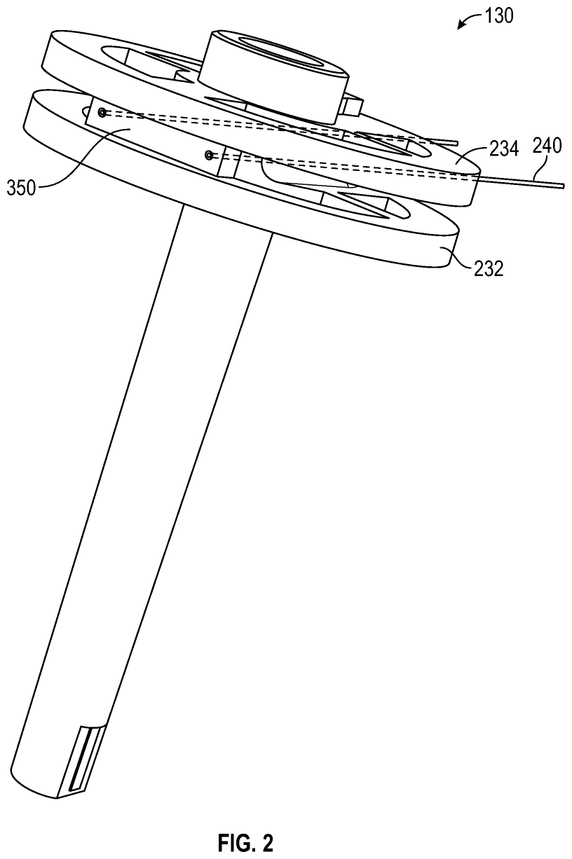

As illustrated in FIG. 2, the rotatable member 130 can be formed from at least a first disc 232 and a second disc 234, similarly shaped and sized, and coaxially spaced a specified distance apart. The first disc 232 and second disc 234 confine the articulation cable 240 (hereinafter “cable”) within the periphery of the rotatable member 130. The first disc 232 can provide a barrier to inhibit or prevent the cable 240 from displacing in an upward direction. The second disc 234 can provide a barrier to prevent the cable 240 from displacing in a downward direction. The dimension of the first disc 232 and the second disc 234 can help limit or minimize the displacement of the cable 240.

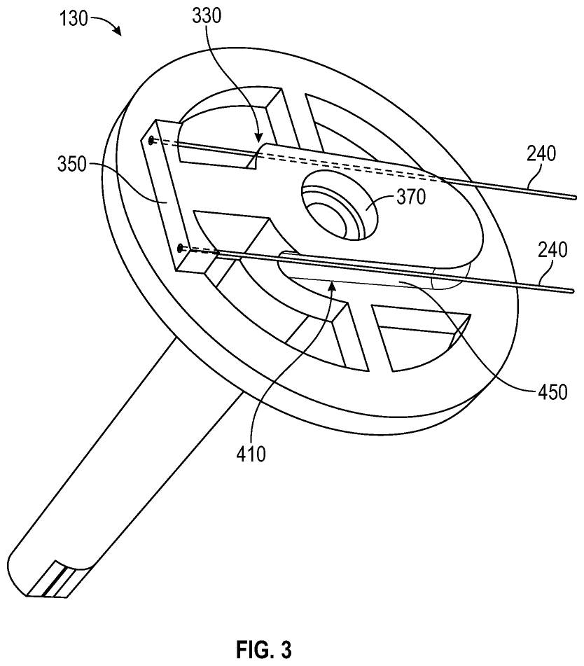

As shown in the example in FIG. 3, the rotatable member 130 has a movable member 330. The movable member 330 can be coupled to a surface of the rotatable member 130. The movable member 330, in an example, protrudes, projects, extends or is otherwise connected to the rotatable member 130. The movable member 330 can project laterally from a surface of the rotatable member 130. For example, the movable member 330 is fixed to the rotatable member 130. When the rotatable member 130 rotates in response to the rotation of the rotatable control member, the rotation is transferred to the movable member 330. The cable 240 is coupled to the rotatable member 130 to, for example, translate movement from the rotatable member 130 to the cable 240 and then to the articulating elongate member 102.

The movable member 330 is, for example, a lever or guide. However, other systems besides lever or guide can be used according to the desired purpose (hereinafter “movable member” will refer to any lever, lever arm or guide). The movable member 330, in some examples, is a hinge point that configured to be rotated with the rotatable member 130. For example, the movable member 330 is a fulcrum about which the movable member 330, as a lever, pivots or rotates. The movable member 330 has grooves, guides, indentations, channels or similar profiles which limits displacement of the at least one articulating cable 240.

The movable member 330 has, for example, a termination coupling 350 remote or located offset or remote from the center 370 of the movable member 330. The termination coupling 350 can also be remote from the center of rotation of the rotatable member 130. The termination coupling 350 can be a stationary coupling that rotates with the rotatable member 130. The termination coupling 350 couples, binds, connects the cable 240 to the movable member 330 and thereby the rotatable member 130. The termination coupling 350 couples the cable 240 by heat staking, clamping, press-fitting, threading, tying, soldering, welding or any other means to couple the cable 240 to the termination coupling 350.

The shape and size of the movable member 330 can be dictated by the desired use of the articulating medical device 100. In an example, the dimension and shape of the movable member 330 maximizes the angular displacement distance of the cables 240 and the articulating member when in operation. In an example, the movable member 330 is at least partially circular, elliptical, cam-like, or other similar shape. As illustrated in FIG. 3, the movable member can be a “U” shape 410 with a substantially arcuate end opposite to the termination coupling 350. As illustrated in FIG. 4, the movable member can be a figure eight shape 420 with the termination coupling 350 at a position remote from the center of rotation of the rotatable member 130.

The movable member 330 has, for example, a channel, path, guide, indentation, on the periphery (hereinafter channel 450) of the movable member 330. In an example, the movable member 330 has a pair of channels 450 extending along opposite sides of the moveable member 330. The channel 450 can limit displacement of the cable 240 when the rotatable member 130 and the movable member 330 rotate. The channel 450, for example, constrains or limits displacement the cable 240 when under slack. The channel 450 can also retain the cable 240 within the circumference of the rotatable member 130. When the cable 240 is retained within the circumference of the rotatable member 130, entanglement of the cable 240 with other features within the cavity 120 of the handle 110 is minimized. The shape of the channel 450 and the shape of the movable member 330, jointly, can minimize entanglement of the cable 240 during operation.

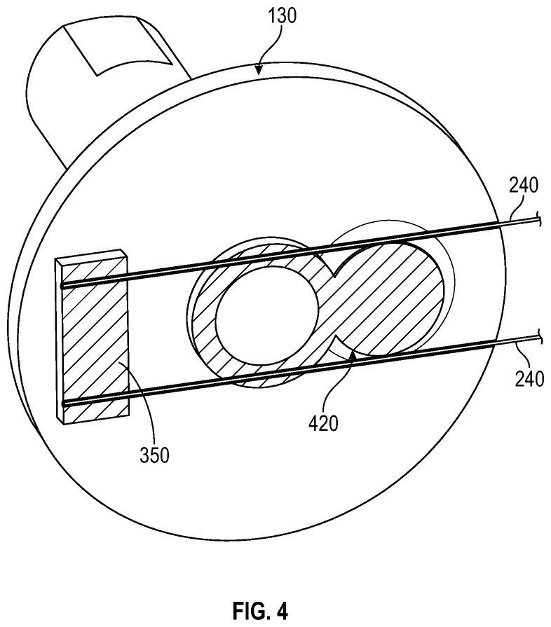

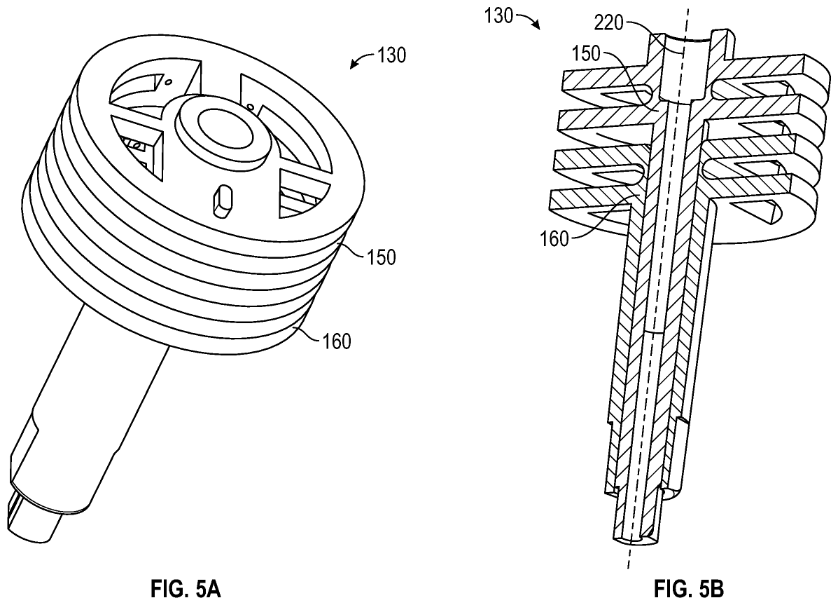

As illustrated in FIGS. 5A and 5B, the rotatable member 130 can include one or more separate rotatable members 130 that are stacked and share a common axis, or coaxially stacked, of rotation 220. For example, the rotatable member 130 includes a first rotatable member (or pulley system) 150 and a second rotatable member (or pulley system) 160 (hereinafter a rotatable member can include disc, reel, spool or pulley) with the shaft of the first rotatable member 150 enclosed within the second rotatable member 160 such that they are coaxially stacked. The rotatable member 130 and associated components within the handle 110 transfer movement to the articulating elongate member 102. In an example where there are two or more separate rotatable members, there are two planes of articulation—horizontal and vertical.

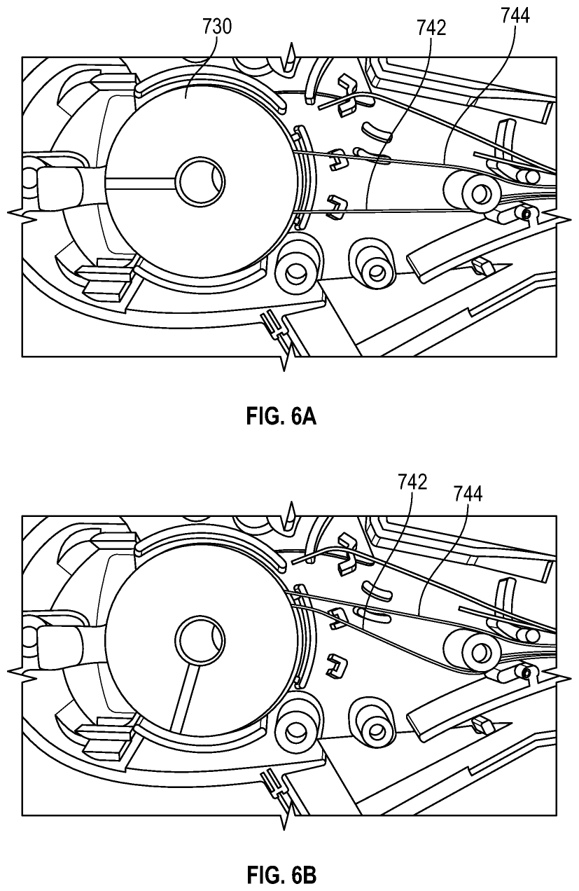

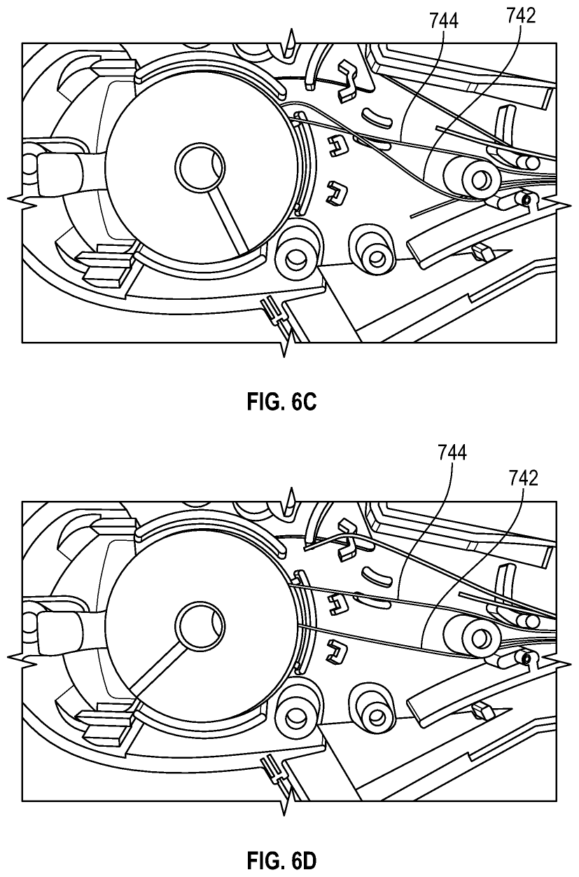

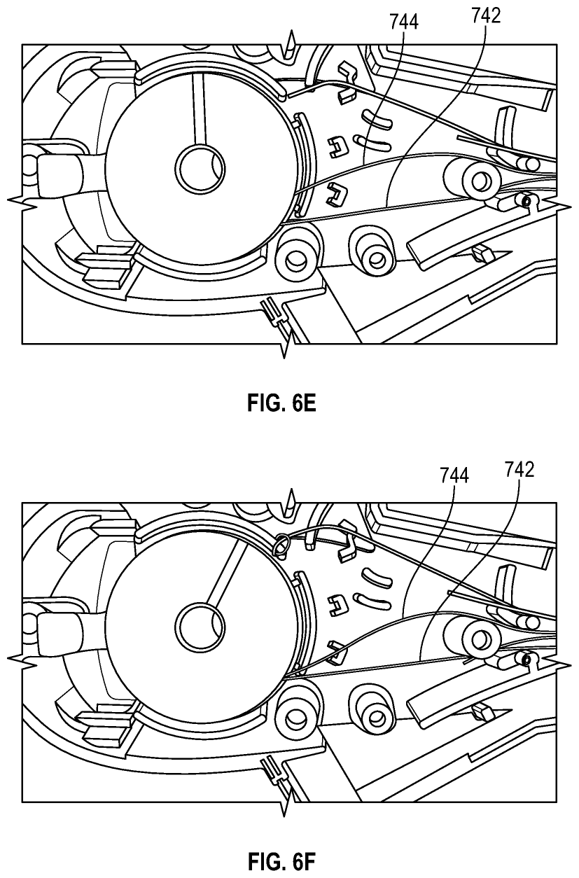

In an example shown in FIGS. 6A-6F, a user manipulates a rotatable control mechanism. The rotation of the rotatable control mechanism translates movement to the rotatable member 730. As the rotatable control mechanism is rotated, the rotatable member 730 rotates in a reciprocal movement. When the rotatable member 730 rotates, the first articulation cable 742 (hereinafter “first cable”), translates or moves in a reciprocal motion about the axis of rotation of the rotatable member 730. The first cable 742 is retained within the guide within the rotatable member 730 such that the guide limits displacement of the first cable 742. The first cable 742 coupled to the termination coupling moves to create tension or slack in a proximal end of the first cable 742. Tension or slack in the first cable 742 occurs within the housing between the movable member within the rotatable member 730 and the articulating endoscopic component. As illustrated in the example movement of the rotatable member in FIGS. 6A-6F, the proximal end of the first cable 742 remains within the circumference of the rotatable member 730 as the first cable 742 is moved. In a slack condition, as shown in FIGS. 6B and 6C, the cable 742, as shown in FIGS. 6E and 6F and the second articulation cable 744 (hereinafter “second cable”) are under slack. The second cable 744 is retained within the guide within the rotatable member 730 such that the guide limits displacement of the second cable 744 in slack. In the example, the cables 742, 744 by being constrained in separate channels avoid being tangled together.

As the first cable 742 moves, motion is translated to the articulating elongate member. In an example shown in FIGS. 6B and 6C, the first cable 742 is in a position of slack and the second cable 744 is in a position of tension. The movable component coupled with the rotatable member 730 allows the first cable 742 and the second cable 744 to be translated to the articulating elongate member without being entangled with any other component or feature within the cavity of the handle.

When either the first cable 742 or the second cable 744 is in a position of slack, the placement of the respective cable within the channel, for example, causes the slack to bow minimally outward and away from the center of rotation of the rotatable member. In another example, the slack is retained or confined to the profile of the rotatable member 730.

In an example the placement and position of the cable, retained within the channel, is controlled. For example, the first cable is attached to a first lateral side of a termination point and the second cable is attached to a second lateral side of the termination point. Further, the first cable can be within the first channel and the second cable can be within a second channel on an opposing side of the axis of rotation. Both the first and second channels are disposed on laterally opposite sides of the axis of rotation of the rotatable member. In this example arrangement minimizes entanglement between the first the second cable. The first cable and the second cable, in an example, move the articulating elongate member in a horizontal direction or a vertical direction according to the specified purpose by creating tension or slack in the cables.

In an example where a second rotatable member is positioned stacked with the first rotatable member and sharing a common axis of rotation, as illustrated in FIGS. 5A and 5B, a third cable and fourth cable extends from a termination coupling of a second movable member. The third and fourth cables extend through a third channel and a fourth channel. The second rotatable member is, for example, rotates in a reciprocal direction according to the rotation of a second rotation control mechanism. When the second rotatable member, and the second movable member, rotate, the third cable and the fourth cable, in an example, move the articulating elongate member in a horizontal direction or a vertical direction different from the first and second cables and according to the specified purpose by creating tension or slack in the cables.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An articulating medical device comprising:
a handle;
an articulating elongate member extending from the handle;
at least one first rotatable member, in a cavity within the handle, the at least one first rotatable member including:
a first lever arm; and
a first termination coupling; and
at least one first articulation cable, coupled to the first termination coupling, wherein the first lever arm is arranged to limit displacement of slack in the at least one first articulation cable when the at least one first articulation cable is not under tension.

2. The articulating medical device of claim 1, further comprising:
a second rotatable member including:
a second lever arm; and
a second termination coupling; and
at least one second articulation cable, coupled to the second termination coupling, wherein the second lever arm is arranged to limit displacement of slack in the at least one second articulation cable when the at least one second articulation cable is not under tension.

3. The articulating medical device of claim 2, wherein the at least one first rotatable member is coaxially stacked with the second rotatable member.

4. The articulating medical device of claim 2, wherein the second articulation cable translates one of horizontal or vertical movement with respect to the articulating elongate member.

5. The articulating medical device of claim 1, wherein the at least one first articulation cable translates one of horizontal movement or vertical movement with respect to the articulating elongate member.

6. The articulating medical device of claim 1, wherein the first lever arm further comprises:
a channel on opposing sides of the first lever arm extending from the first termination coupling towards the articulating elongate member.

7. The articulating medical device of claim 1, wherein the at least one first articulation cable is coupled to the first termination coupling by at least one of threading, heat staking, or welding.

8. A device for controlling movement of endoscopic components, the device comprising:
a handle having at least one external rotatable control mechanism;
an articulating elongate member extending from the handle; and
at least one rotatable member in a cavity within the handle;
wherein the at least one rotatable member includes:
a guide projecting from a surface of the rotatable member and extending laterally from the at least one rotatable member towards the articulating elongate member;
a channel within opposing sides of the guide;
a termination coupling projecting from the surface of the rotatable member and located offset from the guide; and
at least one articulation cable coupled to the termination coupling at a proximal end and extending within the channel towards the articulating elongate member; and
wherein the guide limits displacement of slack in the at least one articulation cable when the articulation cable is not under tension.

9. The device for controlling movement of endoscopic components of claim 8 further comprising:
a second rotatable member, disposed within the cavity, the second rotatable member including:
a second guide projecting from a surface of the second rotatable member and extending away from the second rotatable member towards the articulating elongate member;
a second channel extending along opposing sides of the second guide;
a second termination coupling projecting from the surface of the second rotatable member and located offset from the guide; and
at least one second articulation cable coupled to the second termination coupling at one end and extending within the second channel towards the articulating elongate member;
wherein the second guide limits displacement of slack in the at least one second articulation cable when the articulation cable is not under tension.

10. The device for controlling movement of endoscopic components of claim 9, wherein the second articulation cable translates movement to the articulating elongate member in one of horizontal or vertical direction.

11. The device for controlling movement of endoscopic components of claim 9, wherein the at least one rotatable member is stacked coaxially with the second rotatable member.

12. The device for controlling movement of endoscopic components of claim 8, wherein the at least one articulation cable translates movement to the articulating elongate member in one of horizontal or vertical direction.

13. The device for controlling movement of endoscopic components of claim 8, wherein the termination coupling is offset from the guide on the rotatable member.

14. The device for controlling movement of endoscopic components of claim 8, wherein the guide has an at least partially circular shape.

15. The device for controlling movement of endoscopic components of claim 8, wherein the guide has an at least partially elliptical shape.

16. The device for controlling movement of endoscopic components of claim 8, wherein the handle has at least one exterior control knob coupled with the at least one rotatable member.

17. An endoscopic device having an articulation component extending from a handle, the device comprising:
a first pulley system within a cavity within the handle;
a movable member, coupled to a surface of the first pulley system, including:
a termination coupling remote from a center of rotation of the first pulley system; and
a first pair of channels extending along opposing sides of the movable member;
at least one cable coupled on one end with the pulley system at the termination coupling and extending within one of the pair of channels towards the articulation component; and
wherein the first pair of channels is arranged to limit displacement of slack in the at least one cable when the cable is not under tension.

18. The endoscopic device of claim 17, further comprising:
a second pulley system within the cavity within the handle;
a second movable component coupled to a surface of the second pulley system including:
a second termination coupling remote from the center of rotation of the second pulley system; and
a second pair of channels extending along opposing sides of the second movable component; and
at least one second cable coupled on one end with the second pulley system at the second termination coupling and extending within at least one of the pair of second channels towards the articulation component;
wherein the second pair of channels is arranged to limit displacement of slack in the at least one second cable when the at least one second cable is not under tension.

19. The endoscopic device of claim 18, wherein the second pulley system is coaxially stacked with the first pulley system;
a rotatable control mechanism on an exterior surface of the handle transfers rotation with respect to at least one of the first pulley system and the second pulley system; and
the at least one cable includes at least one first cable, the first cable translates movement with respect to the articulating component in a horizontal direction and the second cable translates movement with respect to the articulating elongate member in a vertical direction.

20. The endoscopic device of claim 17, wherein the movable component includes a substantially arcuate end opposite to the termination coupling.
